(19) United States
(12) Patent Application Publication
(10) Pub. No.: US 2017/0021010 A1
(43) Pub. Date: Jan. 26, 2017

(54) ALLERGY PREVENTING DOSAGE CONTROLLED FOOD PACKETS

(71) Applicant: David Phillip ERSTEIN, Flushing, NY (US)

(72) Inventor: David Phillip ERSTEIN, Flushing, NY (US)

(21) Appl. No.: 14/804,565

(22) Filed: Jul. 21, 2015

(51) Int. Cl. A61K 39/35; B65D 77/02; B65D 75/52

(52) U.S. Cl. CPC A61K 39/35; B65D 75/527; B65D 77/02; A23L 1/30; A23L 1/36; A61K 2039/55

(57) ABSTRACT

A kit of individual packets of food containing a dosage amount of an allergen. The kit has a plurality of packets associated with each other and containing a carrier food in each packet and an allergen so arranged that the respective allergen quantity in each successive packet is larger than in the previous packet. Preferably, the allergen quantities are different and range in weight from 100 mg to 1800 mg in 7 packets. Preferably, each kit has about the same total weight which is in the range of about 3 mg to 4 mg. The contents of each package can be fed to an infant by being dissolved in mother's milk, formula and the like. After the initial introduction packets, maintenance packets, preferably containing 2 mg of the allergen can be fed to the child.

### BACKGROUND OF THE INVENTION

The present invention is generally directed to treatment of allergies and, more particularly, to the preventive treatment of the peanut allergy by early introduction of infants to the allergen and to Oral Immunotherapy (OIT) of infants and/or young children.

Parents who have children suffering from the peanut allergy are constantly gripped with the fear that their child might unwittingly ingest a foodstuff containing a peanut ingredient despite all efforts to prevent it. The consequences can be severe, even life threatening. Peanut allergies affect a significant percentage of young children in Europe and the United States and unlike other common food allergies, e.g., to Hen's Egg, resolution is difficult. As noted, the quality of life of the affected families is reduced because of constant fear over food choices and possibly even the likelihood of anaphylaxis.

It is an object of the present invention to provide a dietary introduction and maintenance product line that concerns and has as its main aim the prevention of allergy consequences by desensitizing children at a very young age to the allergenic foods. The main objective is to gradually introduce non-allergic children as young as 4 months old to a parent's most anxiety-inciting foods. The invention aims to provide a pure organic product line mirrored with strategic diagnosis and distribution in a manner that eases, facilitates and uses very reliable modes of administering the product line to the very young children.

The literature is aware of the general scope of the problem and has provided various solutions thereto, including as reflected in the disclosures of U.S. patent publications 2014/0093541 and 2015/0086593, the contents of which are incorporated herein by reference.

As noted in the literature, peanut allergy is an increasingly troubling global health problem, which affects between 1-3 percent of children in many westernized countries. Although multiple methods of measurement have been used and specific estimates differ, there appears to be a sudden increase in the number of cases in the past 10-15 years period, suggesting that the prevalence may have tripled in some countries, such as in the USA. Extrapolating the currently estimated prevalence, this translates to nearly 100,0000 new cases annually (in the USA and UK), affecting some 1 in 50 primary school-aged children in the USA, Canada, UK and Australia. A similar rise in incidence is now being noted in developing countries, such as Ghana.

The purpose of this disclosure is to highlight emerging evidence to existing guidelines regarding potential benefits of supporting early, rather than delayed, peanut introduction during the period of complementary food introduction in infants. The recent study, entitled “Randomized Trial of Peanut Consumption in Infants at Risk for Peanut Allergy (Learning Early About Peanut—LEAP Trial)”, demonstrated a successful 11%-25% absolute reduction in the risk of developing peanut allergy in high-risk infants (and a relative risk reduction of up to 80%) if peanut was introduced between 4 and 11 months of age. In light of the significance of these findings, this disclosure serves to better inform the decision-making process for healthcare providers regarding such potential benefits of early peanut introduction.

In the aforementioned LEAP trial, 640 high-risk UK infants between the ages of 4 to 11 months were randomized to consume peanut products at least three times a week (6 g of peanut protein; equivalent to 24 peanuts or 6 teaspoons of peanut butter per week) or to completely avoid peanut products for the first five years of life. This included 542 infants found to have negative skin prick tests (SPT) to peanut at study entry, and 98 infants with SPT wheal diameters to peanut butter between 1 and 4 mm (minimally SPT positive) at study entry. An additional 76 children were excluded from study entry prior to randomization based on SPT≧5 mm, which was assumed to have a very high likelihood of reacting to a peanut challenge. In an Intention-To-Treat (ITT) analysis, 17.2% in the peanut avoidance group compared to 3.2% in the peanut consumption group developed food challenge-proven peanut allergy by age 5 years, corresponding to a 14% absolute risk reduction, a number needed to treat (NNT, e.g., number of persons needed to be treated for one to received benefit of 7.1, and a relative risk reduction of 80%.”

When examined in further detail, the isolated beneficial effects for both the primary and secondary prevention of peanut allergy translated to a NNT=8.5 within the SPT negative and NNT=4 within the minimally SPT positive infants. Secondary analyses also showed similar levels of prevention in White, Black and Asian (Indian and Pakistani) children. Overall, the risk of early introduction in this group was low—7 of the 319 children randomized to the consumption group reacted to peanut at the baseline food challenge suggesting that peanut food challenges and introduction, even in minimally SPT positive infants, is safe and flexible. Six children in the consumption group developed peanut allergy during the study, indicating that peanut allergy can still develop despite attempts at primary and secondary prevention. Finally, the LEAP trial only included high-risk infants with a minimal or negative SPT to peanut, and therefore did not address a strategy for those without these risk factors for developing peanut allergy.

Existing guidelines pertaining to the early introduction of complementary foods have indicated that the introduction of highly allergenic foods, such as peanut, need not be delayed past 4 or 6 months of life. However, they do not actively recommend introduction of peanut butter between 4-6 months of age in high-risk infants, and some of these guidelines specify that those infants considered at risk for the development of allergic disease are strongly recommended to first consult an expert.

The LEAP data provide Level 1 evidence that the practice of early peanut introduction is safe and effective in selected high-risk infants. This study is the first prospective, randomized trial of early peanut intervention, and informs provider decision-making regarding high-risk infants, including those already with a positive peanut SPT but not yet clinically reactive, to receive the benefits noted in the LEAP study, which may reduce the risk of developing peanut allergy up to 80%.

Of note, since children with lesser risk factors for peanut allergy were excluded from enrollment in LEAP, there are no prospective, randomized data investigating the benefit or risk of early peanut introduction in the general to low-risk populations. However, multiple guidelines have not recommended delaying allergen introduction in these populations. On this basis, the present disclosure aims to help integrate the findings learned in the LEAP trial to other similar low and high-risk children in more diverse settings internationally.

Based in data generated in the LEAP trial and existing guidelines, the following guidance is suggested to assist the clinical decision-making of healthcare providers.

There is now scientific (Level 1) evidence from a randomized controlled trial that healthcare providers should recommend introducing peanut-containing products into the diet of “high-risk” infants early on in life (between 4-11 months of age) in countries where peanut allergy is prevalent, since delaying the introduction of peanut may be associated with an increased risk of developing peanut allergy.

Infants with early-onset atopic disease, such as severe eczema, or egg allergy in the first 4-6 months of life may benefit from evaluation of by an allergist or physician trained in management of allergic diseases in this age group to diagnose any food allergy and assist in implementing these suggestions regarding the appropriateness of early peanut introduction. Evaluation of such patients may consist of performing peanut skin testing and/or in-office observed peanut ingestion, as deemed appropriate following discussion with the family. The clinician may perform an observed peanut challenge for those with evidence of a positive peanut skin test to determine if they are clinically reactive before initiating at-home peanut introduction. Both such strategies were used in the LEAP study protocol.

Adherence in the LEAP trial was excellent (92%) with infants randomized to consume peanut ingesting a median of 7.7 g peanut protein (interquartile range: 6.7-8.8 g)/week during the first two years of the trial compared to a median of 0 g in the avoidance group. While the outcome if the LEAP regimen was excellent, the study does not address use of alternative doses of peanut protein, minimal length of treatment necessary to induce the tolerogenic effect, or potential risks of premature discontinuation or sporadic feeding of peanut.

Despite the demonstrated efficacy of these preventive treatments, it is still so that the prior art practice of this treatment suffers from the lack of a systematic approach to assure the proper administration of the correct dosages to very young children and the failure of the field to provide better packaged and marked product lines that facilitate the parent's (or responsible adult's) administration of these treatments.

### SUMMARY OF THE INVENTION

The foregoing and objects of the invention may be realized, in accordance with preferred embodiments of the invention by kit of individual packets of food containing a dosaged amount of an allergen, the kit comprising a plurality of packets associated with each other and containing a carrier food in each packet and an allergen, so arranged that the respective allergen quantity in each successive packet is larger than in the previous packet. Preferably, the allergen quantities are respectively about 100 mg, 300 mg, 600 mg, 900 mg, 1200 mg, 1500 mg and 1800 mg. Further preferably, each of the packets are connected to each other by a web material that can be easily torn or broken. In another embodiment, the packets are provided in a box, and so packed in the box that upon withdrawal of the packets from the box, the packets are successively presented in the order of increasing dosage.

Preferably, the allergen is a peanut product and the carrier product and the allergen in each of the packets has a total weight which is about equal in all of the packets. The total weight is preferably in the range of about 3 mg to 4 mg. The carrier food and the allergen are preferably dissolvable in liquid.

Other features and advantages of the present invention will become apparent from the following description of the invention, which refers to the accompanying drawings.

### BRIEF DESCRIPTION OF THE DRAWINGS

FIG. 1 is a product introduction flowchart of the present invention.

FIGS. 2 and 2a show a sample product line in the form of a kit of prepackaged allergy desensitizing ingredients that can be used to attain the objectives of the present invention.

FIG. 3 is a system block diagram of an online, interactive system that facilitates the administration of the food packets or sachets in accordance with the present invention.

### DETAILED DESCRIPTION OF EMBODIMENTS OF THE INVENTION

As described in the aforementioned U.S. 2014/0093541 patent publication, peanuts (Arachis hypogaea) contain multiple allergenic proteins, including Ara h1 to Ara h9 (see for example, Sicherer S H et al J Allergy Clin Immunol. 2007; 120:491-503, de Leon et al Expert Rev Mol Med 2007 9 (1) 1-18). An individual with a peanut allergy may be hypersensitive to one or more of these allergenic peanut proteins. Patients who are hypersensitive to any peanut allergen or combination of peanut allergens may be treated using the methods described below. A patient with a peanut allergy may display peanut-specific serum IgE, i.e. IgE which specifically binds to peanut protein.

Patients may be diagnosed with peanut allergy according to standard clinical criteria. Standard clinical criteria may include for example, a history of a type-1 hypersensitivity reaction which is temporally related to peanut ingestion (e.g. hives, swelling, wheezing, abdominal pain, vomiting, breathlessness), and the presence of peanut-specific IgE by positive skin prick test (wheal diameter>/=3 mm) or ImmunoCap serum IgE>0.35 kU/l. The methods herein may be used for any patient with peanut allergy and are independent of the patient's sensitivity or challenge threshold to peanut allergen, the weight or height of the patient and other factors.

Peanut protein is the total protein contents of a peanut and contains all allergenic peanut proteins, including Ara h1 to h9. Peanut protein may be administered in the form of a whole or part peanut, or it may be extracted, isolated and/or purified from a peanut. For example, peanut protein may be provided as a peanut extract, such as peanut flour.

Peanut flour is produced by crushing, grinding and/or milling whole peanuts. The flour may be partially or completely defatted to reduce the fat content. Defatting does not affect the allergenic peanut protein content of the flour. The peanut protein content of peanut flour may be readily determined using standard techniques and is typically 50% (w/w) peanut protein. Peanut flour is widely available from commercial sources (e.g. Golden Peanut Company GA USA). Other peanut extracts which contain peanut protein may also be used in the methods herein. In some embodiments, total peanut protein may be isolated and/or purified from other constituents of peanuts for use.

In some embodiments, peanut protein may be administered as a whole peanut, e.g., to adults. This may be preferred, for example, for high incremental doses of peanut protein, such as 400 mg and 800 mg. Peanut protein represents 25% (w/w) of a peanut and the average weight of a peanut is 500-650 mg. An incremental dose of 400 mg may be administered as 2-3 large peanuts, and an incremental dose of 800 mg may be administered as five large peanuts. Suitable whole peanuts include any form of roasted peanut, including salted and honey roast, and coated or embedded peanuts, for example peanuts coated or embedded in a food product, such as chocolate or yogurt. In some embodiments, peanuts which constitute the incremental dose of peanut protein may be crushed and presented inside a food product, such as a small biscuit, cake, chocolate, sweet or jam.

Conveniently, peanut protein, optionally in the form of peanut flour, may be mixed with a carrier to produce a composition for administration to the patient. Suitable carriers mask the peanut protein from the mouth and upper gastrointestinal (GI) tract and reduce or prevent local itching/swelling reactions in these regions during administration. For example, a carrier may contain one or more lipid, polysaccaride or protein constituents. The carrier may be a food product, for example a dairy or dairy substitute product, such as yogurt, milkshake or chocolate, or another food product with similar properties. Dairy substitute products may include soy-based products. In some embodiments, the composition for administration may be a food product which has been supplemented with peanut protein, for example in the form of peanut flour. The composition may be any food product which can be produced with a discrete dose of peanut protein, e.g. chocolate, yogurt, confectionery (e.g. sweets and jellies) or beverages. In some embodiments, the composition may be a cooked or baked food product, such as a biscuit or cake. The peanut protein may be added at any stage of the production of the food product.

The food product may be supplemented with flavorings to mask the taste of the peanut protein. Suitable food flavorings are well known in the art and include sugar, mint, vanilla and orange essence. The food product may be supplemented with preservatives, stabilizing agents, fillers, colorings and sweeteners in accordance with standard food production techniques.

In other embodiments, the composition for administration may be an oral delivery vehicle such as a capsule, cachet or tablet, each of which contains a predetermined amount of peanut protein to provide the correct incremental dose to the patient. Oral delivery vehicles may be useful, for example, in avoiding contact between the peanut protein and the mouth and upper gastrointestinal tract. Suitable carriers, binders, fillers or diluents lubricants and preservatives for use in oral delivery vehicle are well known in the art.

In some embodiments, the composition for administration may further comprise other components, for example, anti-allergy drugs, such as antihistamines, steroids, bronchodilators, leukotriene stabilizers and mast cell stabilizers. Suitable anti-allergy drugs are well known in the art. This may be useful in reducing allergic inflammation and increasing tolerance of the peanut protein.

As described below, compositions, such as food products, for use as described herein may be formulated in unit dose formulations which contain a defined amount or amounts of peanut protein.

In accordance with the present invention, the drawbacks of the prior art can be ameliorated or avoided altogether by providing packaged products in kit form which contain allergy-preventing ingredients such as peanuts and various tree-nuts (almonds, walnuts, cashews, etc.). Different food items are intended to be sold separately. Each package introduction kit contains the pure, uncontaminated food product in incrementally increasing dosages. The medium of food introduction is preferably a powder designed to be easily mixed into foods (carrier agents) that are known to be tolerated specific to the child (breast milk, formula, cereal, applesauce, etc.). The child will start with small dosages and upon subsequent feeds and days, a doubling of amounts (or different qualifier) is administered until a defined target rate per volume dose/protein content to be used as maintenance and continued exposure over time.

These packaged products are provided in easy to open packets or containers and in a form ready to mix. As an example, the product will be sold as a package with several packets within, ranging in size from 0.1 g to 2.0 g (e.g., 0.3, 0.6, 0.9, 1.2, 1.5 and 2.0 g). This kit containing these seven packets is administered over seven days, after which maintenance packets that contain individual packets with 2.0 g of peanut are recommended to use every week. The maintenance packets can be packaged to contain eight individual packets or sachets or two months of continuous maintenance, or other packet quantities. The maintenance packets are recommended to be used until the child can tolerate the food in spread or nut form.

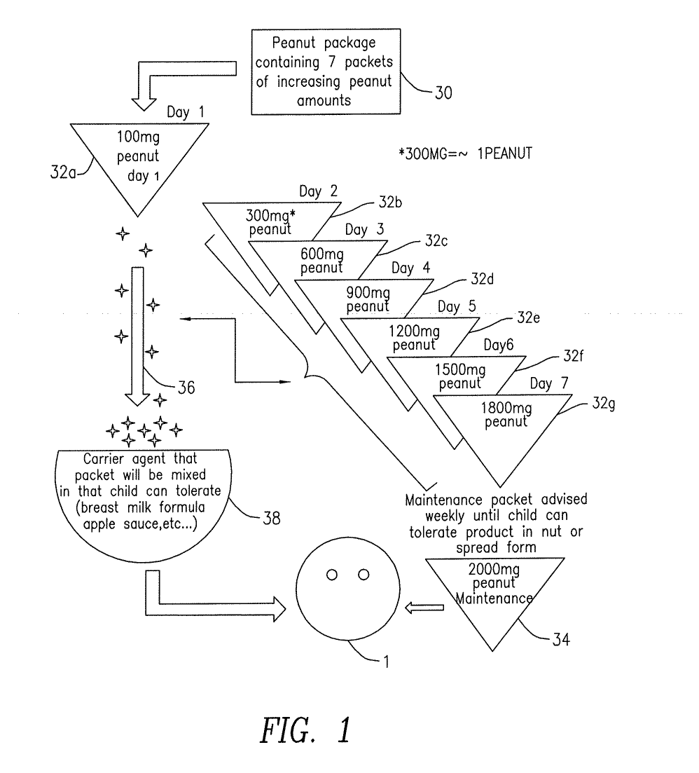

FIG. 1 is a flowchart that illustrates the mode of administering the products of the invention. In its salient configuration, the present invention comprises a peanut package 30 containing a number of packets of increasing peanut amounts, preferably seven packages. These packages of increasing peanut amounts, identified herein as packets 32a through 32g, are intended to be administered to the infant throughout day 1 through day 7, with the amount of contained peanut starting with 100 mg and then increasing to the amounts 300 mg, 600 mg, 900 mg, 1200 mg, 1500 mg and 1800 mg, as indicated in the figure. Each of the packages can have a total weight of 3 mg to 4 mg, which includes the carrier. This mixture can be easily mixed with the child's food, which may be breast milk, formula, apple sauce, etc., as indicated at 38. The tiny amount of peanut allergen indicated by the arrow 36 is administered to the child on successive days. Once the child has reached the 1800 mg peanut amount, the maintenance packets may be separately provided and administered weekly until the child can tolerate the peanut product in nut or spread form.

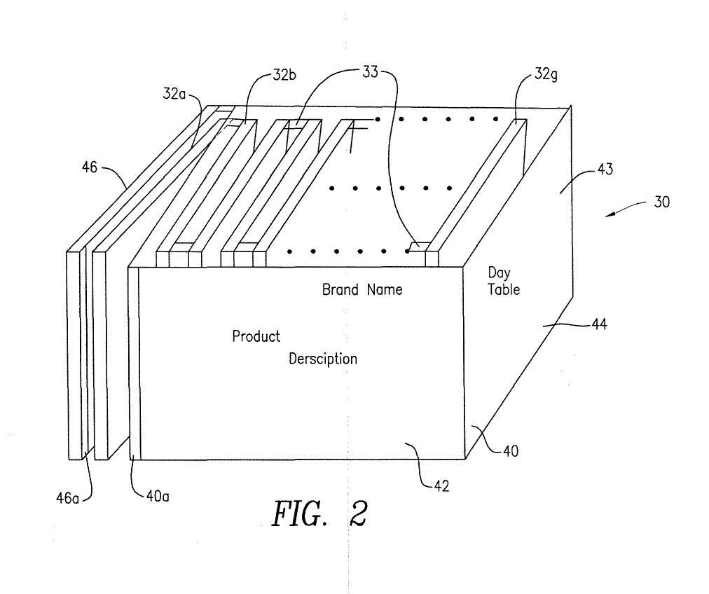

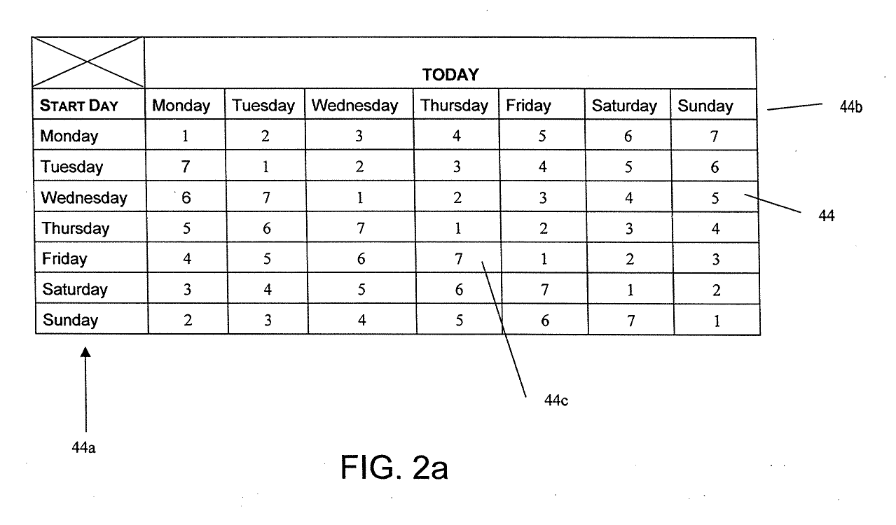

Referring to FIG. 2, the products 30 of the present invention can be packaged in a box 40 that has one face 42 printed with information such as brand name, product description, and other information. The box 40 has an interior space 43 containing the packets 32a, 32b, 32c, 32d, 32e, 32f and 32g as shown. The interior space 42 can be closed by a lid 46 that has at one edge a magnetic strip 46a which is attracted to a corresponding magnetic strip 40a on the box 40.

Another face of the box 40 may have a day table 44. This table is easy to consult as shown in FIG. 2a, and can be in the form providing one column 44a identifying the start day, i.e., Monday, Tuesday . . . Sunday, and other columns 44b identifying the day being consulted to determine which packets to administer. This is merely a safety table that allows a mother or responsible adult who knows that the infant treatment has begun, for example, on Friday, to then immediately determine that on Thursday it should be packet number 7 that needs to be administered. Rather obviously, because extreme care has to be taken not to provide too large a dosage too soon, one would not wish a parent to become confused and administer a particular packet out of sequence.

Regardless, as an added precaution, the bags shown in FIG. 2 are so packed as individual bags that they are all connected to each other by tearable or detachable webs, so that the individual packets would be retrieved from the box 40 only in the correct order. Still, the table of FIG. 2 provides the added safety because it reminds a parent that they may have forgotten to administer the packet of a particular date, and the table would cause them to reflect whether they should not contact their doctor concerning how to proceed, particularly if they have forgotten the sequence for a number of days. In the above example, if the packet for day 7 is identified by reference numeral 44c.

As noted above, the invention is intended to provide an introduction kit, as well as maintenance kits for non-allergic infant populations. These packets, whether the introduction or maintenance packets, contain a mixture of sprouted oats and peanut flour. With a relatively stable total weight of approximately 3-4 g for each packet, there will be increasing amounts of peanut flour for each subsequent day in the package, with equal decreasing weight of the carrier sprouted oats. A great benefit of using a sprouted oats and peanut flour blend is that the mixture is easily dissolvable in liquid at room temperature. It can be noted that the sprouted oats have more health benefits than traditional oats.

The product is completely organic and contains no other ingredients aside from oats and peanuts although the product invention can be prepared with substitute for the oat component, for example, similar grains such as barley.

The kit's sachets are semi-attached and ordered in such a way that one cannot confuse the correct order of recommended sachet ingestion. The outer packet can have a scratch-off pad which shows the day packets that have already been administered. The kit can be in a box/tin that will contain detailed instructions on proper use of the introduction kit and maintenance kit.

The invention can be also provided to include additional ingredients; for example, by adding probiotics to the peanut/oat blend, as studies have demonstrated that probiotics have been helpful in desensitizing those individuals who are already peanut allergic. Other optional constituents are pre-made products such as yogurts, applesauce, etc., with similar increasing introductory blends that are suffused with the product of the invention for both the introduction and maintenance blends.

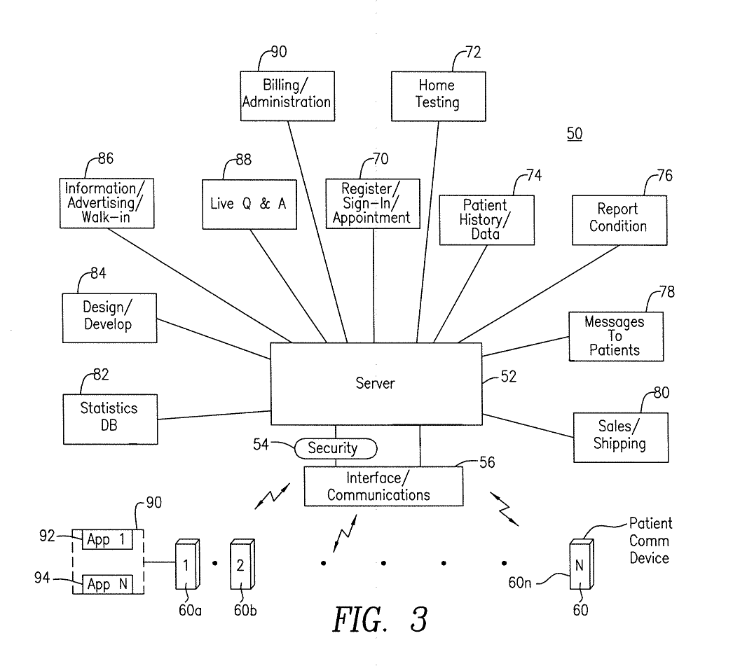

The versatility and usefulness of the present invention is further enhanced by providing an online computer facility 50 which is block diagrammed in FIG. 3. Referring to FIG. 3, the system comprises a main server 52 with an interface/communication hardware 56 that communicates either wirelessly or via the Internet or by any known communication means with patients' or potential patients' communication devices 60, numbered 60a, 60b . . . 60n in the figure. These patient communication devices 60 may be handheld devices such as a mobile phone or the desktop computer or the like, in well known manner. Whether parents or concerned adults, they can always access the website which comprises the front-end software of the server 52. If these interested people who wish to communicate are already patients and have patient files, they would be able to communicate via the security block 54 and thereby reach their confidential account information. Or, they can communicate with the server generally for information and data concerning the services being provided and formalities such as how to become a registered patient and the like.

Patients would typically register or sign in or make doctor appointments of the software block 70. Parents can also communicate with the server 52 to consult their patient history or data at block 74, which provides a history of their child in the treatment program or just historic information or other relevant information. In another facility, the server allows reporting conditions of a child at block 76 or even provide information using equipment that administers tests and those tests results being provided through the equipment and thereby being recorded at the home testing block 72. Certain patients may be afforded or permitted live questions and answers at block 88, depending on the type of questions, whether an emergency, etc.

In response to a report of a condition at block 76, the system automatically sends messages to patients at block 78 either by text message, email, voice messages and other communications means well known in the art.

The server 52 may also handle sales and shipping at block 80, so that patients may be approved for or may themselves decide to buy packets that they require for their child. For example, the information/advertising/walking block 86 may provide information on different carriers or formulations for the same dosage packages.

Lastly, the blocks 82 and 84 are configured to run software that gathers statistics about responses of the infant population to the various dosages based on various criteria such as the age when the treatment has begun, geographical location, gender and other relevant information and the program and the medical researchers responsible may be involved in the design and development of new products and new dosages, new allergen carriers, all in the quest of treating more infants earlier in life to prevent allergies from developing in the first instance.

The server 52 may also include software modules 90 responsible for handling billing and/or administrative tasks necessary to assure the well running of the overall treatment program. Further, the individual patient communication devices 60 may be provided with various APPs, including an APP 92 that accesses the server 52 at the touch of an icon or an APPn 94 that allows a responsible adult to follow the administration of the treatment packets to each particular child.

While the invention has been being described herein relative to peanut allergies, it can be similarly applied to deal with other allergenetic foods; for example, peanuts, almonds, cashews, walnuts, pecans, hazelnuts, etc., all in a similar method/manner.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A kit of individual packets of food containing a dosaged amount of an allergen, the kit comprising:
a plurality of packets associated with each other and containing a carrier food in each packet and an allergen, so arranged that the respective allergen quantity in each successive packet is larger than in the previous packet.

2. The kit of claim 1, wherein the allergen quantities are respectively about 100 mg, 300 mg, 600 mg, 900 mg, 1200 mg, 1500 mg and 1800 mg.

3. The kit of claim 1, wherein each of the packets are connected to each other by a web material that can be easily torn or broken.

4. The kit of claim 3, wherein the packets are provided in a box, and so packed in the box that upon withdrawal of the packets from the box, the packets are successively presented in the order of increasing dosage.

5. The kit of claim 1, wherein the allergen is a peanut product.

6. The kit of claim 5, wherein the carrier product and the allergen in each of the packets has a total weight which is about equal in all of the packets.

7. The kit of claim 6, wherein the total weight is in the range of about 3 mg to 4 mg.

8. The kit of claim 1, wherein the carrier food and the allergen are dissolvable in liquid.

9. The kit of claim 1, wherein the carrier food comprises one or more of peanuts, almonds, cashews, walnuts, pecans and hazelnuts.
